(19) United States
(12) Patent Application Publication
(10) Pub. No.: US 2003/0065218 A1
(43) Pub. Date: Apr. 3, 2003

(54) Process for preparing sorbic acid from sorbic acid polyester

(76) Inventors: Christoph Mollenkopf, Frankfurt (DE); Erwin Weiss, Hofheim (DE); Thomas Beck, Idstein (DE); Andreas Schneider, Koenigstein (DE); Klaus Fahrner, Kelkheim (DE); Stefan Purps, Koenigstein (DE)

Correspondence Address: ProPat, L.L.C., 2912 Crosby Road, Charlotte, NC, 28211-2815, US

(21) Appl. No.: 10/237,364

(22) Filed: Sep. 9, 2002

(30) Foreign Application Priority Data
Sep. 21, 2001 (DE) 101 46 689.7

(51) Int. Cl. C07B 35/04; C07C 57/10

(52) U.S. Cl. 562/601; 562/599

(57) ABSTRACT

The invention relates to a process for preparing sorbic acid by cleaving the sorbic acid polyester prepared from crotonaldehyde and ketene, the sorbic acid polyester being distilled and the cleavage being catalyzed by an amine, which comprises separating off the amine from the distillation residue by distillation under reduced pressure and at a temperature which is higher than the temperature of the polyester distillation and recovering it.

### BACKGROUND OF THE INVENTION

Various processes are known for preparing sorbic acid. A particularly economical process starts from the polymeric polyester reaction product which is prepared by reacting crotonaldehyde with ketene in an inert solvent in the presence of a fatty acid salt of a divalent and/or trivalent metal of subgroups II to VIII of the Periodic Table of the Elements as catalyst (DE-A-10 42 573).

Sorbic acid can be produced in various ways from this polyester.

An industrially important process consists of thermal catalytic cleavage of the polyester which comprises cleaving the polyester in the presence of an inert diluent which boils at atmospheric pressure above 150° C., preferably above 180° C. (DE-A-10 59 899) and 0.5% to 50% of a secondary or tertiary amine boiling at atmospheric pressure above 100° C., preferably above 150° C., as catalyst at temperatures of 160° C. to 220° C., simultaneously distilling off the sorbic acid and the diluent (DE-A-12 82 645). Particularly suitable solvents are the aliphatic carboxylic acids of appropriate boiling point specified in DE-A-10 59 899.

When this process is carried out industrially, the cleavage is carried out in a continuous distillation apparatus. The sorbic acid polyester dissolved in the diluent is charged into the distillation vessel where the amine-catalyzed cleavage of the sorbic acid polyester to give sorbic acid takes place. The sorbic acid formed is distilled off together with the diluent via a rectification column at 160-200° C. and 20-50 hPa with reflux. Rectification is necessary in order to prevent the transfer of amine into the distillate and to achieve the appropriate purity.

The sorbic acid is then crystallized out of the distillate and separated off from the diluent. The diluent is recirculated.

In parallel to the desired cleavage of the sorbic acid polyester to give sorbic acid, a decarboxylation reaction of the sorbic acid polyester takes place to give carbon dioxide and pentadiene, and thus decreases the yield. This reaction can largely be suppressed by increasing the amine content in the bottom-phase of the cleavage. Thus, for example, an approximately 4% higher yield of sorbic acid is obtained when the amine concentration in the bottom-phase of the cleavage is increased from 10% to 40%.

Since in the thermal cleavage of sorbic acid polyester in the distillation vessel, in addition to sorbic acid, polymers are also formed which do not distill under these conditions and lead to an increase in the bottom phase, these continuously formed polymers must constantly be discharged as residue. Since the catalyst amine is mixed with the polymers, catalyst amine is also unavoidably co-discharged from the bottom, so that to maintain the amine concentration in the bottom-phase of the cleavage, fresh amine must constantly be added.

To quadruple the concentration of the catalyst amine in the bottom phase, in order to achieve the increase in yield in cleavage of the sorbic acid polyester, the amine feed must also be approximately quadrupled. Since this amine must be discharged again together with the polymer from the bottom, however, this also means quadrupling the amount of amine as residue.

It was therefore an object to develop a process in which the catalyst amine is recovered from the residue and thus can be reused for repeated use as catalyst for cleavage of sorbic acid polyester.

### BRIEF DESCRIPTIONS OF THE INVENTION

The invention therefore relates to a process for preparing sorbic acid by thermal cleavage of the polyester prepared from crotonaldehyde and ketene in which, from the inevitably produced residue, the amine used as catalyst is recovered and can thus be reused. This first provides the possibility of increasing the amine concentration in the bottom-phase of the cleavage to achieve a higher yield of sorbic acid without amine being lost. Secondly, as a result, the use of fresh amine can be reduced. In addition, this is accompanied by a reduction in the total amount of residue.

### DETAILED DESCRIPTION OF THE INVENTION

Surprisingly, it has been found that by distilling the residue at 190 to 220° C., preferably 205 to 215° C., and at a pressure of 5 to 15 hPa, preferably 7 to 9 hPa, the catalyst amine can be selectively separated off from the other bottom-phase constituents.

Advantageously, the process can be carried out in a thin-film evaporator. Particularly good results are obtained when a tertiary amine is used as catalyst, in particular a trialkylamine having two C₁-C₃-alkyl groups, in particular methyl groups, and an alkyl chain having 14 to 20 carbon atoms, in particular 15 to 17, very particularly preferably 16 carbon atoms.

Suitable diluents to carry out the cleavage of the sorbic acid polyester are aliphatic, alicyclic, aromatic hydrocarbons, their chlorine, bromine and nitro derivatives, and also ethers and silicone oils whose boiling point at atmospheric pressure is above 150° C., preferably above 180° C. However, ketones, esters, carboxylic acids and alcohols having the appropriate boiling range can also be used as diluents, although in general the results are not quite as good, since they apparently in part react with the reaction mixture. It is expedient to use those diluents or solvents which are liquid at ambient temperatures, boil at atmospheric pressure below 300° C., preferably below 270° C., and form azeotropic mixtures with sorbic acid, so that they at the same time act as entrainers, such as petroleum fractions, dodecane, tetradecane, 5-methyldodecane, dodecene, dicyclohexylmethane, p-di-tert-butylbenzene, 1-methyinaphthalene, 2-methylnaphthalene, 1-ethylnaphthalene, tetrahydro-naphthalene, diphenylnaphthalene; halogenated aliphatic, cycloaliphatic or aromatic hydrocarbons such as dichlorododecane, 1,5-dibromopentane, benzotrichloride, o- and m-dibromobenzene; nitro compounds such as nitrobenzene, 2-nitrotoluene; nitrites such as benzyl cyanide; carbonyl compounds such as acetophenone or the heterocyclic 2-acetylthiophene; heterocyclic compounds such as chromane, thiophene; ethers such as resorcinol dimethyl ether, diphenyl ether, safrole, isosafrole; acids such as enanthric acid, α-ethylcaproic acid, caprylic acid, capric acid; or esters such as ethyl benzoate, methyl phenylacetate and methyl salicylate.

The examples below illustrate the invention.

The starting material is a polyester-containing reaction product which was obtained in a similar manner to DE-B-10 42 573, example 1. In this method 420 g of ketene are introduced at a temperature between 25° C. and 35° C. into a stirred mixture of 800 g of crotonaldehyde, 1200 ml of toluene and 14.2 g of zinc isovalerate. The excess crotonaldehyde and the toluene are removed in vacuo. The residue obtained is 1150 g of polyester in the form of a high-viscosity brown liquid. In addition to the zinc content of 3000 ppm, this reaction product still contains fractions which cannot be converted into hexadienoic acids, such as diketene polymers and crotonaldehyde resins.

The proportion convertible into hexadienoic acids was determined by basic saponification of a solution of 60 g of sorbic acid polyester in 120 g of toluene using 33 g of potassium hydroxide in 260 g of water at room temperature. This produces in the aqueous phase potassium sorbate and the potassium salt of 3-hydroxy-4-hexenoic acid, from which hexadienoic acid can be produced by acidification. The proportion of the polyester which can be converted into hexadienoic acids can be determined by quantitative determination of the two reaction products by means of HPLC.

Under these mild conditions, the polyester content can be determined much more accurately than as described in DE-A-12 82 645. Thus the proportion of the crude polyester which is convertible into hexadienoic acids is 89 to 90% and not, as assumed in DE-A-12 82 645, only 80%. The yields achieved in DE-A-12 82 645 must therefore be corrected, see example 1 (comparative example).

### EXAMPLE 1

### Comparative Example

The apparatus consists of a 1 l 3-neck round-bottomed flask (reaction flask) having an attached distillation column. The distillation column, of a filling height of 600 mm and an internal diameter of 40 mm, is packed with glass Raschig rings 6 mm in diameter. The distillation column bears a column top cooled to 70° C. with a reflux splitter. The reflux splitter firstly recycles condensed distillate to the column, and secondly passes it for collection in a graduated heatable receiver (500 ml) and a 6 l round-bottomed flask. The entire apparatus is operated under vacuum, and an oil pump with an upstream dry ice cold trap generates the vacuum.

This apparatus is operated semibatchwise, and to achieve statistically meaningful results, a plurality of experiments are carried out reusing the filtrate and bottom-phase liquid produced in the respective preliminary experiment.

In the first experiment, 260 g of a mixture consisting of 12% dimethylhexa-decylamine and 88% Arkopal® (=nonylphenol polyglycol ether as residue liquefier) are placed in the reaction flask.

The apparatus is evacuated to about 30 hPa and the reaction flask is heated with the oil bath (bath temperature approximately 220° C.). When the temperature in the reaction flask reaches 180° C., the feed mixture is metered (417 g/h) into the reaction flask at a reflux ratio of 1.

The feed mixture for the reaction flask consists of 350 g of polyester (see above), 2128 g of ethylhexanoic acid, 12 g of dimethylhexadecylamine and 10 g of Arkopal® (=nonylphenol polyglycol ether as residue liquefier) (total amount 2500 g).

After the feed mixture has been metered in, pure 2-ethylhexanoic acid is run through the apparatus without polyester and without reflux (834 g) for 2 hours and then redistilled for 5 min. The distillate situated in the receiver is homogenized by heating to 50-55° C. and then cooled in the course of 3 hours to 20° C. with stirring (500 rpm). After this temperature has been reached, the mixture is kept for a further 15 min at 20° C. and then the crystallized crude sorbic acid is filtered off with suction and the pure content determined by gas chromatography.

In this experiment the bottom phase in the reaction flask increases by 62 g (starting from 260 g). This increase in residue consists of 12 g of dimethylhexadecylamine, 10 g of Arkopal, 2 g of sorbic acid and 2-ethylhexanoic acid and 38 g of sorbic acid polymer and is discharged from the system.

In each further experiment, after separating off the crude sorbic acid, the filtrate is used in the feed mixture, instead of the pure 2-ethylhexanoic acid, together with 350 g of polyester, 12 g of dimethylhexadecylamine and 10 g of Arkopal.

Then, 260 g of the bottom phase from the respective preliminary experiment is placed in the reaction flask, which bottom phase has a mean dimethylhexa-decylamine concentration of 12%.

After the experiment has been carried out a number of times, a mean sorbic acid yield of 74% is obtained. Based on the pure polyester, that is solely taking into account the proportion of 90% which can be cleaved to form hexadienoic acids, a yield of 82.2% is thus calculated.

### EXAMPLE 2

The sorbic acid polyester cleavage procedure is carried out as in example 1 (comparative example). In the first experiment, 260 g of a mixture consisting of 40% dimethylhexadecylamine and 60% Arkopal® (=nonylphenol polyglycol ether as residue liquefier) are placed in the reaction flask.

The feed mixture for the reaction flask consists of 350 g of polyester (from example 1), 2128 g of 2-ethylhexanoic acid, 48 g of dimethylhexadecylamine and 14 g of Arkopal (total amount 2540 g).

The bottom phase in this reaction increases by 104 g. This increase in residue consists of 48 g of dimethylhexadecylamine, 14 g of Arkopal, 4 g of sorbic acid and diluent and 38 g of sorbic acid polymer and must be discharged from the system before the next experimental procedure.

The filtrate, after separating off the crude sorbic acid, is reused in the feed mixture in the following experiment, instead of the 2-ethylhexanoic acid, together with 350 g of polyester, 48 g of dimethylhexadecylamine and 12 g of Arkopal.

Then, 260 g of the bottom phase from the respective prior experiment is placed in the reaction flask, which bottom phase has a mean dimethylhexadecylamine concentration of 40%.

After the experiment has been carried out a number of times, a mean sorbic acid yield of 79.9% is obtained. Based on the pure polyester, that is to say only taking into account the proportion of 90% which can be cleaved to form hexadienoic acids, a yield of 88.7% is thus calculated.

### EXAMPLE 3

The sorbic acid polyester cleavage procedure is performed as in example 2.

As in example 2 the bottom phase in this reaction increases by 104 g. This increase in residue consists of 48 g of dimethylhexadecylamine, 14 g of Arkopal, 4 g of sorbic acid and 2-ethylhexanoic acid and 38 g of sorbic acid polymer and must be discharged from the system before the next experimental procedure.

In a thin-film evaporator, this discharged bottom-phase residue is distilled at 210° C./8 hPa. For a thin-film evaporator heating area of 16 cm², a throughput of 450 g/h is possible. The rotor equipped with movable scraper blades has a peripheral velocity of 3 m/s. 205 g/h of distillate and 245 g/h of residue discharge. The starting amount of 104 g produces 47 g of distillate and 57 g of residue.

The sorbic acid polymers and the liquefier Arkopal, in addition to small amounts of dimethylhexadecylamine, are present in the thin-film evaporator effluent. The majority of the ejected amine (43 g) and 4 g of sorbic acid and 2-ethylhexanoic acid are present in the distillate.

This distillate is supplemented with 5 g of fresh dimethylhexadecylamine to 48 g of total amine and reused in a similar manner to example 2 in the next cleavage experiment together with the diluent from the crude sorbic acid separation, 350 g of polyester and 12 g of Arkopal.

After the experiment had been carried out a number of times with recirculation of the amine a mean sorbic acid yield of 79.9% is obtained. Based on the pure polyester, that is to say only taking into account the proportion of 90% which is cleavable to form hexadienoic acids, a yield of 88.7% is calculated.

Yields and amine usage of the respective experiments are compared in summary form in the table below:

| | Example 1 | Example 2 | Example 3 |
|---|---|---|---|
| Amine content in the bottom phase | 12% | 40% | 40% |
| Amine recycling | no | no | yes |
| Yield | 82.2% | 88.7% | 88.7% |
| Increase in residue during the | 62 g | 104 g | 104 g |
| reaction | | | |
| “Fresh” amine feed | 12 g | 48 g | 5 g |
| Amine feed from amine recirculation | | | 43 g |
| Residue ejected from the system | 62 g | 104 g | 57 g |

1. A process for preparing sorbic acid by cleaving the sorbic acid polyester prepared from crotonaldehyde and ketene, the sorbic acid polyester being thermally cleaved and the cleavage products being distilled and the cleavage being catalyzed by an amine, which comprises separating off the amine from the distillation residue by distillation under reduced pressure and at a temperature which is higher than the temperature of the polyester distillation and recovering it.

2. The process as claimed in claim 1, wherein the cleavage products are distilled in the presence of a diluent.

3. The process as claimed in claim 2, wherein the diluent is selected from the group consisting of aliphatic, alicyclic, aromatic hydrocarbons, their chlorine, bromine and nitro derivatives, and also ethers and silicone oils.

4. The process as claimed in claim 1, wherein the cleavage products are distilled under reduced pressure at about 20 to about 50 hPa.

5. The process as claimed in claim 1, wherein the cleavage products are distilled under reduced pressure at about 160 to about 200° C.

6. The process as claimed in claim 1, wherein the amine is selected from the group consisting of secondary or tertiary amines having a boiling point at atmospheric pressure above about 100° C., preferably above about 150° C.

7. The process as claimed in claim 1, wherein the distillation residue is distilled under reduced pressure in a thin-film evaporator.

8. The process as claimed in claim 1, wherein the distillation residue is distilled under reduced pressure at about 5-15 hPa in a thin-film evaporator.

9. The process as claimed in claim 1, wherein the distillation residue is distilled under reduced pressure and at a temperature of about 190 to about 220° C. in a thin-film evaporator.

10. The process as claimed in claim 1, wherein about 70 to about 90% by weight of the amine previously contained in the residue is recovered.
